(19) United States
(12) Patent Application Publication
(10) Pub. No.: US 2025/0177158 A1
(43) Pub. Date: Jun. 5, 2025

(54) SMALL BONE ANGLED COMPRESSION SCREW

(71) Applicant: ExsoMed Corporation, Aliso Viejo, CA (US)

(72) Inventors: Lloyd P. Champagne, Phoenix, AZ (US); Jozef Zoldos, Phoenix, AZ (US)

(21) Appl. No.: 19/052,395

(22) Filed: Feb. 13, 2025

Related U.S. Application Data
(63) Continuation of application No. 17/449,542, filed on Sep. 30, 2021, now Pat. No. 12,251,316.
Application No. 17/449,542 is a continuation of application No. 16/122,264, filed on Sep. 5, 2018, now Pat. No. 11,147,681.
(60) Provisional application No. 62/554,130, filed on Sep. 5, 2017.

(51) Int. Cl. A61F 2/42; A61B 17/68; A61B 17/72; A61B 17/86; A61F 2/30

(52) U.S. Cl. CPC A61F 2/4241; A61B 17/7225; A61B 17/7291; A61B 17/8615; A61B 17/8635; A61B 2017/681; A61B 17/8605; A61B 17/863; A61F 2002/30156; A61F 2002/30205; A61F 2002/30224; A61F 2002/30322; A61F 2002/30622; A61F 2002/4243

(57) ABSTRACT

The present invention relates to a device and system for surgical fixation of small bones, small bone fragments, and osteotomies and more particularly to compression screw having a threaded leading portion which is joined to a section that is free from threads, and which includes an angle or from 12° to 25° in a plane through the longitudinal axis of the screw and a portion which is joined to a head having a configuration that is intended to provide anti-rotational stability and compression through the device.

### INCORPORATION BY REFERENCE TO ANY PRIORITY APPLICATIONS

This application is a continuation of U.S. patent application Ser. No. 17/449,542, filed Sep. 30, 2021, which is a continuation of U.S. patent application Ser. No. 16/122,264, now U.S. Pat. No. 11,147,681, filed Sep. 5, 2018, which claims priority from U.S. Provisional Application No. 62/554,130, filed Sep. 5, 2017.

Any and all applications for which a foreign or domestic priority claim is identified in the Application Data Sheet as filed with the present application are hereby incorporated by reference under 37 CFR 1.57.

### FIELD OF THE INVENTION

The present invention relates to a device and system for surgical fixation of small bones, small bone fragments, and osteotomies and more particularly to compression, screw having a threaded leading portion which is joined to a section that is free from threads, and which includes an angle or arc from 12° to 25° in a plane through the longitudinal axis of the screw and a trailing portion which is joined to a head having a configuration that is intended to provide anti-rotational stability and compression through the device.

### BACKGROUND OF THE INVENTION

Patients often suffer from late stage arthritis in phalangeal joints of the hands and feet, and this presents a variety of challenges for attending physicians. While current treatment protocols usually provide acceptable results, there is a likelihood of straight distal interphalangeal joint fusion which provides for sub-optimal outcomes. Research has shown that when a patient's distal interphalangeal joint is fused in a functional position, finger dexterity and grip strength improve over that of a patient with a straight fusion. Physicians can achieve angled fusions by using k-wire fixation, however, this immobilization protocol can fail, and lead to several complications and varied results. While the utilization of compression screws can provide reliable, strong repairs, it does not offer the additional benefit of function flexion which is provide by a properly angled and oriented implant fixation device, especially one, which provides the added benefit of compression across the joint during fusion.

Advantageous locations the use of the present invention is in the phalanges of the hand or foot. In each finger, there are three phalanges that are separated by two joints called the interphalangeal joints (IP joints). The proximal IP joint (PIP joint) is the one closest to the MCP joint. The other joint closest to the end of the finger is the distal IP joint (DIP joint). The thumb just has one IP joint. The joints are covered on the ends with articular cartilage. The foot has an analogous structure substituting the large toe for the thumb. It should be understood that there may be additional surgical techniques or locations in the body where the device of the present invention may be suitable for use.

### SUMMARY OF THE INVENTION

The present invention solves the problems associated with performing a fusion technique or an osteotomy, in particular in the interphalangeal joints. The device of the invention is a compression screw having a leading portion including a beveled and/or fluted self-tapping cutting tip, and a threaded portion, and an intermediate shaft portion which joins the trailing portion and forms an angle of from 12° to 25°, and preferably 18°+/−3°. Moreover, the intermediate shaft portion is joined to a trailing head portion that is configured to provide for compression across the fusion joint, and to inhibit rotation of the compression device in the interphalangeal position. The head is specifically configured (in a modified truncated triangular shape including hemi-cylindrical longitudinal flange members or alternatively, a tapered conical shape having threads) to be inserted into the bone without damaging the bone because it is sized and shaped to support the bone from the inside and to fit in the narrow confines of the intramedullary channel of these bones, to provide a head design which provides compression across the joint, but which can be seated below the surface of the bone to avoid screw prominence on the digit tip or intrusion into the fat pad of the finger or toe and the irritation that can result from a proud portion. In addition, the present invention provides for a percutaneous insertion in a surgical technique with an intramedullary implant designed to minimize soft tissue, cartilage and vascular damage upon insertion; and to facilitate early, active mobilization post-operative protocols for accelerated healing and earlier return to work.

The head at the trailing end includes a driving recess, such as a hexalobe, capable of being driven by a suitable driver into the opening.

### BRIEF DESCRIPTION OF THE DRAWINGS

FIG. 1 is an isometric view of a device in accordance with the invention;

FIG. 2 is a side view of the device of FIG. 1;

FIG. 3 is a side view of the device of FIG. i taken at a rotation of 90° to the view in FIG. 2;

FIG. 4 is a cross-section of the device of FIG. 3 taken along line A-A;

FIG. 5 is a detail of FIG. 1 showing the end view of the head of the device from the top;

FIG. 6 is a dorsal view of a skeleton of a hand showing the implant of the invention in place in a PIP fusion;

FIG. 7 is an illustration of the step of inserting a guide wire in a retrograde fashion to align the distal and intermediate phalanges in accordance with the technique of the invention;

FIG. 8 is an illustration of the step of drilling by passing a cannulated drill over the guide wire;

FIG. 9 is an illustration of the step of inserting and confirming the placement of the implant across the fusion site in accordance with the surgical technique of the invention;

FIG. 10 is an isometric view of a second embodiment of the device in accordance with the invention;

FIG. 11 is an end view of the device of FIG. 10 taken from a first end; and

FIG. 12 is an end view of the device of FIG. i 0 taken from the second end.

### DETAILED DESCRIPTION OF THE INVENTION

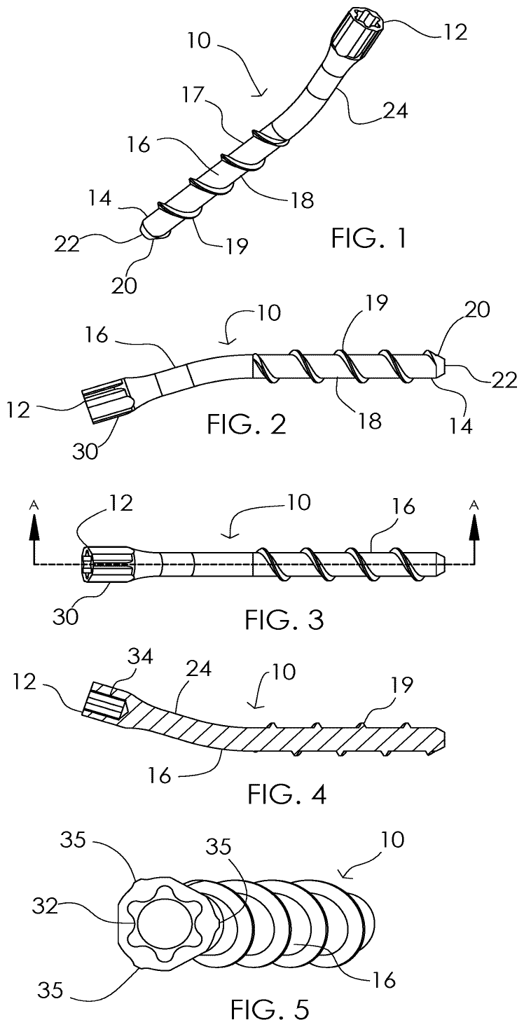

FIG. 1 shows an exemplary embodiment 10 of the angled compression screw of the present invention. The screw 10 may be formed of any suitable biocompatible material, such as surgical grade stainless steel, titanium, alloys of nickel and chromium, nitinol, PEEK, hydroxyapatite, bio-glass or other bio compatible materials or combinations of these materials. The screw 10 has a first end, or trailing end 12, a second end, or leading end 14, a shaft 16 with an outer surface 17, and the shaft 16 including a leading portion i 8 which includes a thread 19 and has a beveled cutting tip 20 at the terminal end 22. The leading portion 18 is joined to a trailing portion 24 of the shaft 16. It should be noted while that the trailing portion may be considered to be proximal to the leading portion relative to the screw itself, in use, the leading portion is intended to be implanted more proximally in the joint than the trailing portion. The leading portion includes an intermediate angled area that joins the leading portion 18 of the shaft to a compression head 30 which has a driving surface 32 in a drive recess 34 formed in the top of first end 12. The angle of the angled area is from 10° to 25°, and preferably 18°+/−3° which is defined at the intersection of the central longitudinal axes of the trailing portion and the leading portions of the shaft. Accordingly, the outer surfaces of the angled portion include a slight radius. The shaft is only angled in a single plane as can be seen in a comparison of FIG. 2 and FIG. 3.

In the first embodiment, the cutting end 20 includes a bevel and a counterbore that helps to cut through any bone left behind when the bone is drilled to receive device 10, and further provides a space to receive extraneous material dragged along during insertion. The driving surface 32 in this embodiment has a hexalobe drive configuration, although any suitable driving configuration may be used. Other driving configurations that may be used include slotted, Pozidriv, Robertson, tri-wing, Torq-Set, SpannerHead, Triple Square and hex head.

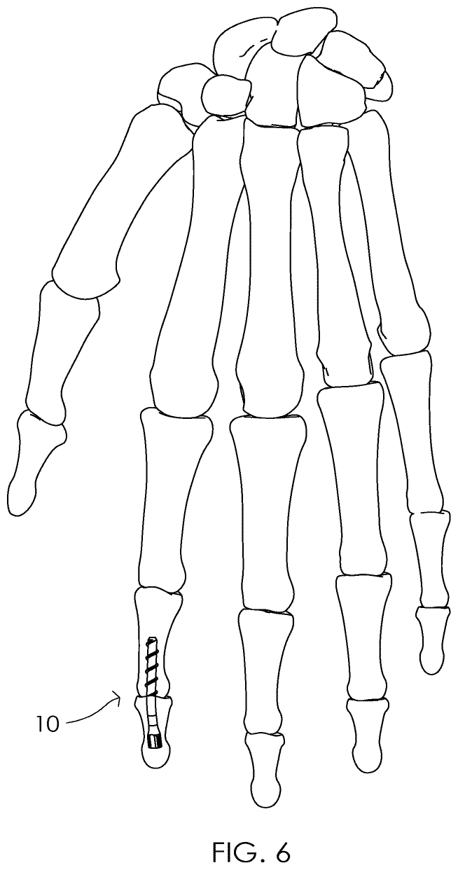

In the first embodiment, the head of the compression screw 30 has a profile as seen from the top in FIG. 5 that represents a truncated modified triangle. In particular, the tips of the angle of the triangle are rounded slightly and instead include hemi-cylindrical flanges 35, that serve to strengthen the head 30 in areas where the torque driving recess has been cut-out, and further which helps to inhibit the distal flange from rotating. The configuration is radially symmetrical geometric shape (here a modified triangle as described below), but irregular, meaning that it is a circle of a diameter that is larger than the radius of the arc prescribed by the bent shaft rotated about a circle, or slightly larger than the size of the intermutually channel. Then, from 2-5 flat surfaces are designed in the circle, and optimally three flats to carve a triangular shape, and the flats can additionally include other protrusions, such as the hemi-cylindrical flanges in order to better wedge the head into position in the intramedullary channel of the distal phalange to secure it in place and inhibit rotation relative to the intermediate flange. Optionally, the terminal area of the head can flare or widen in cross-section in order to enhance the compression.

The leading portion of the device includes a thread 19 which is defined between the outer diameter and the inner diameter and can suitably include a right handed single start thread with a pitch of from 3-4, and preferably at 3+/−0.5 with a similar lead value. The leading and following profiles of the threads together form an angle of 60°+/−15°, and preferably 10°, and with a thread depth of 0.1 mm to 0.4 mm, and optionally a thrust profile or buttress profile.

FIG. 6 illustrates a screw 10 in accordance with the present invention in position across a first PIP joint to secure a fusion.

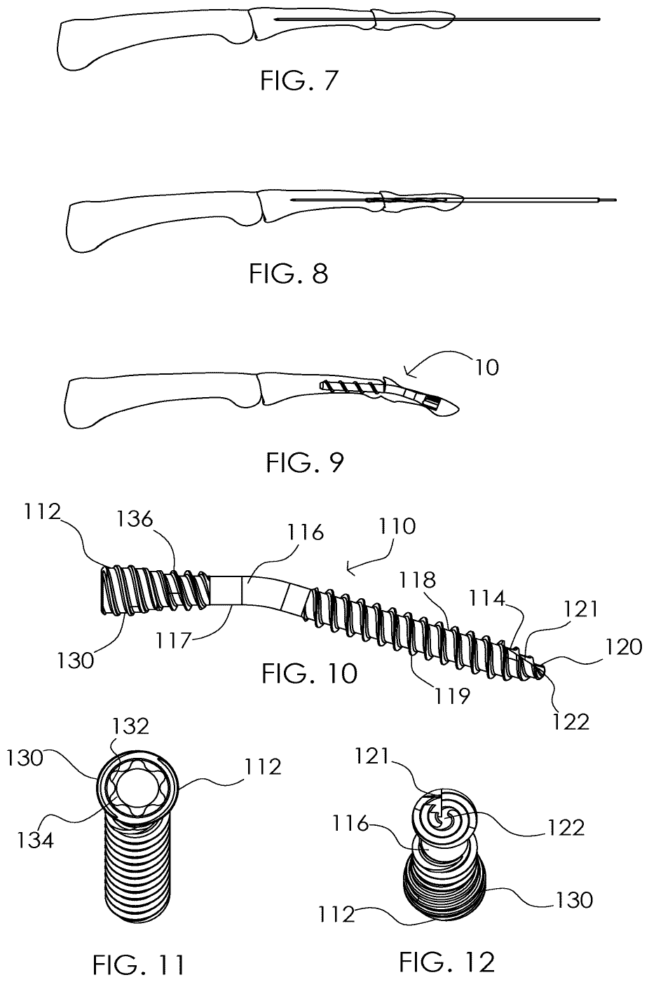

In FIG. 7, in a first step of a surgical technique in accordance with the invention, the joint is scored for fusion and a k-wire is inserted in the intramedullary cannel in retrograde until is abuts the inners cortical surface of the phalanges.

In FIG. 8 a cannulated drill 3, uses the K-wire as a guide to drill an opening (i.e., a straight opening or hole) into the phalanges extending through the fracture and providing enough space on each side of the fracture to properly position device 10.

In FIG. 9, the device i 0 is driven into the opening in the phalanges by means of the drive recess. The outer diameter of the threads 28, is slightly larger than the inner diameter of the opening in the bone This provides bone material for threads 28 to thread into and provides a tight fit for device 10. Further, the device is configured to allow a bent angled screw to be screwed into a straight hole which has been provided in the cancellous portion of the bone and across a fusion site, but where the device includes an angle along its long axis so that the relationship of the bones, or bone segments is changed as the device is screwed into the hole. Specifically, the device includes an angle in one plane about its long axis which is between 10° and 25°, and more specially which is 18°+/−3°. This angle is defined by the intersection of the axes along the central long axis of the device, and further where the device includes a leading length and a trailing length each extending from the intersection to the termination of the device (i.e., the lateral surfaces at the leading tip and the area surrounding the torque driving recess), where the trailing length describes an arc of rotation when it is rotated about the long axis which describes an arc of rotation diameter that is not larger than, or equal to a cross-sectional diameter of the head of the device below any additional trailing compression feature. Again, this means that the head has a configuration in the cross-section taken transverse to the long axis which is a modified regular geometric shape, such as a circle or an oval that has a maximum size that fill not exceed the space in the bone from external cortical surface to external cortical surtace, but which can be “dragged” through the bone in rotation as the threaded leading portion of the screw is screwed into the bone, but which allows the cancellous bone to fill in during healing. Thus, the head has a capability to fill the canal of the bone that is created by the passing of the bent section in order to create compression in addition to having an the anti-rotation aspect which inhibits the device or the bone from rotating from the intended angled orientation (so that an oddly angled phalange is avoided while the desired fusion angle is preserved). This feature is governed by the largest diameter section of the head in contrast to the cuts or flats that create the anti-rotation feature.

It is noted that the head could include a trailing flared portion which exceeds the more leading area in order to increase the feature of compression, and that this trailing flare has a depth along the long axis from the trailing end of the head of less than ½, and more preferably ¼ of the total length of the head. Thus, the head can be dragged into the bone, (which can include a counter-bored portion) without splitting the bone, and will seat below the cortical surface of the bone.

FIGS. 10-12 show a second embodiment of the invention which is similar to the first embodiment, except that the head is a more traditional tapered or conical shape and is threaded in a double lead thread of the same thread shape and pitch value or even a lower pitch value (i.e. 50% to 95%, and preferably 75% to 90% of the pitch value) as the leading portion of the screw. The screw 110 has a first end, or trailing end 112, a second end, or leading end 14, a shaft 116 with an outer surface 117, and the shaft 116 including a leading portion 118 which includes a thread 119 and has a tapered cutting tip 120, including a cutting flute 121 at the terminal end 122. The leading portion 118 is joined to a trailing portion 124 of the shaft 116 The trailing portion includes an intermediate angled area that joins the leading portion 118 of the shaft to a tapered or conical compression head 130 with a thread 136 and which has a driving surface 132 in a drive recess 134 formed in the top of first end 112.

Having thus described some embodiments of the invention, other variations and embodiments that do not depart from the spirit of the invention will become apparent to those skilled in the art. The scope of the present invention is thus not limited to any particular embodiment, but is instead set forth in the appended claims and the legal equivalents thereof. Unless expressly stated in the written description or claims, the steps of any method recited in the claims may be performed in any order capable of yielding the desired result.

What is claimed is:

1. A compression screw for use in an intramedullary channel of a bone, the screw comprising:
a first portion having a first longitudinal axis, the first portion comprising a thread; and
a second portion extending distally from the first portion and having a second longitudinal axis, the second portion including a bend from the first portion,
wherein at least a portion of the second portion includes a thread, the thread of the second portion having a smaller pitch than the thread of the first portion,
wherein the second portion comprises a torque driving recess, and
wherein the screw is non-cannulated.

2. The compression screw of claim 1, wherein the bend forms an angle of from 10° to 25° between the first longitudinal axis and the second longitudinal axis.

3. The compression screw of claim 1, wherein the bend forms an angle of 18°+/−3° between the first longitudinal axis and the second longitudinal axis.

4. The compression screw of claim 1, wherein the torque driving recess comprises a hexalobe.

5. The compression screw of claim 1, wherein the screw is only bent in a single plane.

6. The compression screw of claim 1, wherein an end of the first portion comprises a cutting flute.

7. The compression screw of claim 1, wherein the thread of the second portion has a pitch that is 50% to 90% of a pitch of the thread of the first portion.

8. The compression screw of claim 1, wherein the thread of the second portion has a pitch that is 75% to 90% of a pitch of the thread of the first portion.

9. The compression screw of claim 1, wherein a largest diameter of the second portion is larger than a largest diameter of the first portion.

10. The compression screw of claim 1, wherein the screw is configured to be used in distal interphalangeal joint replacement or fusion.

11. The compression screw of claim 1, wherein the thread of the first portion has a thread depth of 0.1 mm to 0.4 mm.

12. The compression screw of claim 1, wherein the thread of the first portion is a single start thread.

13. The compression screw of claim 1, wherein the bend is nonthreaded.

14. The compression screw of claim 1, wherein at least a portion of the second portion is nonthreaded.

15. The compression screw of claim 1, wherein the second portion comprises a taper from a first diameter to a second diameter.

16. The compression screw of claim 1, wherein the thread of the second portion is a double lead thread.

17. The compression screw of claim 1, wherein the second portion comprises a conical shape with a larger diameter at a free end tapering to a smaller diameter.

18. The compression screw of claim 1, wherein the first portion comprises a tapered cutting tip.

19. The compression screw of claim 18, where in the thread of the first portion extends along the first longitudinal axis from a free end of the tapered cutting tip to an opposite end of the first portion.

20. The compression screw of claim 1, wherein the second portion has a length that is from ¼ to ½ of a total length of the screw.
